(19) United States
(12) Patent Application Publication
(10) Pub. No.: US 2002/0132343 A1
(43) Pub. Date: Sep. 19, 2002

(54) System and method for delivering umbilical cord-derived tissue-matched stem cells for transplantation

(76) Inventor: Clark Lum, Potomac, MD (US)

Correspondence Address: Michelle S. Marks, Shaw Pittman LLP, 1650 Tysons Boulevard, McLean, VA, 22102, US

(21) Appl. No.: 10/090,632

(22) Filed: Mar. 6, 2002

Related U.S. Application Data
(60) Provisional application No. 60/276,476, filed on Mar. 19, 2001.

(51) Int. Cl. C12N 5/08; G01N 33/48; G01N 33/50

(52) U.S. Cl. 435/372; 702/19; 435/2

(57) ABSTRACT

The present invention relates to cord blood stem cell systems, methods, and products for delivering tissue-matched stem cells derived from a single cord blood source, for use in transplantation. The cord blood stem cell system standardizes, customizes and integrates cord blood-derived stem cell expansion to produce progenitor cells to treat hematological and other diseases. The system integrates the production of patient-matched stem cells so that the patient's physician may order the biological product and receive customized cord blood-derived stem cells as an approved biologic ready for transplantation and capable of long term engraftment.

### BACKGROUND

This application claims the benefit of U.S. provisional application 60/276,476, filed Mar. 19, 2001, which is hereby incorporated by reference herein in its entirety.

### FIELD OF THE INVENTION

The present invention relates generally to the field of human stem cell transplantation products, methods and systems. More particularly, the present invention relates to stem cells products derived from cord blood, the therapeutic uses of such stem cells, and more particularly to a system and method for producing a licensed biological product, comprised of tissue-matched stem cells, that is delivered to the clinic where it is administered to the patient. The stem cells of the biological product have been tissue-matched to an individual patient.

### BACKGROUND OF THE INVENTION

The major sources of transplantable hematopoietic cells in adults and children have been the bone marrow and growth factor-mobilized peripheral blood cells. The residual blood left in the placenta and umbilical cord (collectively referred to as “umbilical cord blood” or “UCB”) contains hematopoietic stem and progenitor cells that are of sufficient quantity to engraft a small recipient. Over the past several years, both related and unrelated cord blood has been used as a source of hematopoietic stem cells, particularly in pediatric patients.

Cord blood stem cells (CBSC) are either autologous (i.e., self; the recipient is also the donor) or allogeneic (i.e., intra-species; from a different donor of the same species) products derived from a one-time collection of a limited numbers of cells from placental/umbilical cord blood. The major limitation to the widespread use of UCB for stem cell transplantation has been the inadequate number of stem cells available from a single tissue matched source. The low cell number contributes to delayed engraftment of neutrophils and platelets and limits the use of UCB in adults and larger patients. Studies have shown that the more UCB cells that are transplanted to the recipient, generally the shorter the time to engraftment and, in some cases, the better the outcome of the transplant. Gluckman et al., Cord blood hematopoietic stem cells: biology and transplantation. In: Hematology. W. B. Saunders, pp 1-14 (1998).

In order to produce more cord blood cells from a single source, methods for expanding the cord blood cells ex vivo are being developed. Although methods for ex vivo expansion of UCB cells have been described, such methods have not established that the expanded UCB cells are capable of rapid, complete, and sustained long-term hematopoietic reconstitution after transplantation in adult humans. See, for example, Broxmeyer et al., PNAS USA 89:4109 (1992); Durand et al., Leuk Lymphoma 11:263 (1993); Moore et al., Blood Cells 20:468 (1994); Kogler et al., Bone Marrow Transplant 21:233 (1998); Denning-Kendall et al., Bone Marrow Transplant 21:225 (1998); Koller et al., Bone Marrow Transplant 21:653 (1998) and Qiu, et al., J. Hematotherapy Stem Cell Res, 8:609 (1999).

Also, for UCB expansion to be used clinically, the cells need to be cultured in media that is free of bovine or human serum and is capable of supporting the proliferation and differentiation of the stem cells, such as CD34+ cells. Early progenitor/stem cells express the cell differentiation marker known as CD34+. CD34+ cells can differentiate into all the different hematopoietic linages in the presence of specific cytokines. The term “serum-free” means that whole serum is excluded from the medium, although certain purified or recombinantly produced serum components can be added to the medium.

Over the past few years, transplant clinics have struggled with managing the development and delivery of expanded UCB stem cells of consistent quality and predictable engraftment outcome. For example, tissue-matched and expanded UCB stem cells are not typically delivered to a transplant center in a form that is ready for administration to the patient. Typically, the transplantation center produces the expanded stem cells locally using approved devices and ingredients for the expansion process. For example, Aastrom Corporation markets a bioreactor for a transplant center to use to expand stem cells, which is described in U.S. Pat. Nos. 6,096,532; 6,048,721; 5,994,129; 5,985,653; 5,688,687 as well as Van Zant et al., Blood Cells 20:482 (1994); Koller et al., Bone Marrow Transplant 21:653 (1998) and at www.aastrom.com. Also, the University of Minnesota produces expanded cord blood for its transplant program. See http://server3.cancer.umn.edu.

Several steps are required to manufacture an expanded stem cell product, each of which is typically carried out by a separate establishment. For example, one establishment may recover tissue from cord blood, another establishment may make the donor-suitability determination, a third may process the tissue, and a fourth may distribute the product. No system has integrated the following manufacturing steps that are needed for the successful production of an individualized yet standardized stem cell product: (i) determining donor-suitability, (ii) cell processing and (iii) product distribution. These unintegrated systems do not fully manage the entire life cycle of UCB stem cell production, regulatory licensure and delivery to the distant clinic that administers the cells to the patient. Many clinicians and transplant centers with patients in need of stem cell replacement therapy, therefore, are foregoing the use of expanded UCB as a source of stem cells and turning to lesser desirable sources of replacement cells or sending the patient to distant locations where stem cells may be available.

Accordingly, no one has sufficiently managed the life cycle of customized expanded UCB stem cell production, regulatory licensure and delivery to the clinic. For the treatment of patients in need of hematopoietic stem cells and other cellular therapeutics, there is a need for an integrated system and method for managing the entire life cycle of UCB stem cell production, licensure and delivery that can be accessed by clinicians regardless of their location and local availability of stem-cell expansion capabilities. There is a need for an effective method of managing the production, regulatory approval, and delivery of stem cell therapeutics that places the stem cell product in the context of other approved biologics that have defined labeling regarding efficacy, safety and purity.

### SUMMARY OF THE INVENTION

Embodiments of the present invention relate to systems and methods for stem cell production, regulatory approval and delivery. A Structured Cord Blood System (SCBS) includes the collection, production, regulatory licensure and delivery of UCB stem cells. A SCBS delivery system manages the regulatory approval of each customized stem cell product and delivers a patient matched, FDA approved product to the location where it is to be administered to the patient.

The Structured Cord Blood System (SCBS) of the present invention is capable of producing sufficient numbers of tissue-matched cord blood stem cells (“SCBS products”) for transplantation in adults, from a single donor umbilical cord blood source. The SCBS has the potential for producing sufficient numbers of the cord blood-derived progenitor cells for multiple engraftments in clinical use. The SCBS of the present invention standardizes, customizes and integrates cord blood-derived stem cell expansion to produce donor progenitor cells for use in allogenic stem cell transplantation in adults. The SCBS also includes delivery systems that contain the required stem cell populations for treating hematological and other diseases. SCBS products can be administered to humans in order to prevent, treat, cure, diagnose, or mitigate disease or injury. The cell products to be generated by the SCBS are used to treat diseases that respond to the introduction of stem cells and progenitor cells, as well as expanded and differentiated cells for specific tissue types. Therefore, the SCBS can be used to treat diseases such as cancer, with or without preceding myeloablative chemotherapy, where new and vital tissues are introduced to the patient. The SCBS can also be used to correct genetic diseases, where human tissues are incapable of producing a functional cell, such as in sickle cell anemia. Yet another application of the SCBS of the present invention is in the production and introduction of viable tissues to treat humans having such diseases and disorders as aplastic anemia, liver cirrhosis, diabetes and neurodegenerative processes (e.g., Parkinson's, amyotrophic lateral sclerosis and stroke) .

The SCBS products of the instant invention are capable of obtaining product licensure from the FDA (i.e., FDA approval) and other health authorities in other countries and regulatory territories, as well as product labeling with characterizing information regarding product indication, product efficacy, safety and purity. The SCBS facility of the instant invention is capable of obtaining establishment licensure from the FDA ) and other health authorities in other countries and regulatory territories.

It is an object of the present invention to provide a stem cell manufacturing system for delivering tissue-matched stem cells, the system comprising: a delivery system for delivering a biological product comprised of tissue-matched stein cells and a stem cell expansion system that produces a biological product, said stem cell expansion system being coupled to the delivery system. Another object of the present invention is to provide a stem cell manufacturing system that includes a delivery system and stem cell expansion system conducted at the same licensed establishment. A further object of the present invention is to provide a stem cell manufacturing system in which the stem cell said biological product is ready for administration to the patient.

Yet another object of the invention is to provide a stem cell manufacturing system in which the delivery system receives the patient order, and identifies a suitable source of donor blood. Yet another object of the present invention is to provide such a stem cell manufacturing system in which the donor blood is umbilical cord blood or any other source of stem cells, including allogeneic stem cells.

Another object of the stem cell manufacturing system of the present invention is that the tissue-matched stem cells are CD34+ cells, matched by HLA-A, HLA-B, and HLA-DR loci. It is a further object of the present invention to provide a stem cell manufacturing system in which the tissue-matched stem cells are CFU-GM cells. Another object of the stem cell manufacturing system of the present invention is to provide a licensed biological product. The tissue-matched stem cells are matched using DNA-based testing methods and/or low resolution/split antigen level and preferably the stem cells possess at least three HLA loci identical to the HLA loci of the patient order.

The SCBS production methods include processes for expanding umbilical cord-derived CD34+ stem cells from a single umbilical cord source in serum-free media, such as QBSF-60. In one embodiment of the present invention, the CD34+ cells are expanded more than 200-fold. The SCBS of the present invention includes a process for expanding CD34+ stem cells from a single umbilical cord source. In one embodiment of the SCBS invention, the CD34+ cells are expanded about 200-fold and/or produces about 100 million CD34+ stem cells.

In another embodiment of the present invention, the at least 100 million CD34+ stem cells are produced from a single umbilical cord source. In another embodiment of the present invention, the SCBS process produces at least a 200-fold expansion of CFU-GM and/or generates at least 20 million CFU-GM. In another embodiment of the present invention, the SCBS process produces at least a 200,000-fold expansion of the LTC-IC. In another embodiment of the present invention, the SCBS process produces at least a 60-fold expansion of BFU-E/CFU-Mix.

### BRIEF DESCRIPTION OF THE DRAWINGS

FIG. 1 is a schematic diagram showing the cord blood stem cell production system of the present invention and the life cycle of a structured cord blood system product of the present invention.

FIG. 2 is a schematic diagram showing processes within the structured cord blood system of the present invention.

FIG. 3 is a schematic diagram showing one example of the structured cord blood expansion process including one example of the structured cord blood closed container system.

### DETAILED DESCRIPTION OF THE INVENTION

The Structured Cord Blood System and Product Life Cycle

Embodiments of the present invention relate to systems and methods for managing the production cycle of customized stem cells, such as cord blood derived stem cells, including the development, management, maintenance and delivery of stem cells. Stem cells (SC) may include, without limitation, progenitor cells and stem cells capable of repopulating the hematopoietic system, as well as other mesenchymal tissues.

The SCBS provides standard operating procedures (SOPs) to document and process all phases of the SCBS life cycle. The SCBS preferably includes an accredited, licensed SCBS facility and licensed SCBS products. For example, the SCBS life cycle may be comprised of phases that preferably include activities to (1) match patient immunology to an UCB source and/or an allogenic tissue source; (2) receive source material and confirm patient immunology match; (3) purify the desired stem cell subpopulation(s); (4) expand the desired stem cells ex vivo; (5) produce a Patient Treatment Kit (PTK) ready for administration; and (6) ship the PTK to the transplantation center for its administration to the patient. Each phase of the life cycle comprises a plurality of procedures.

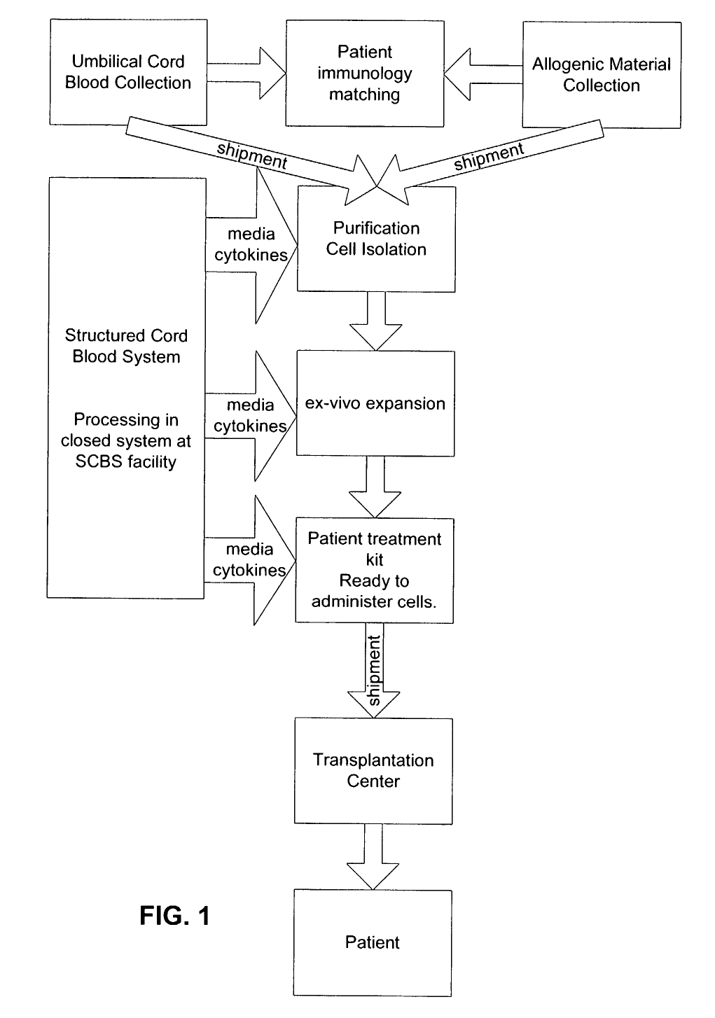

FIG. 1 is a schematic diagram showing the SCBS system and the life cycle of a SCBS product. The SCBS process may be broadly defined as an integrated program to manage the life cycle of UCB stem cell production, licensure and delivery of an FDA approved stem cell therapeutic product. The Structured Cord Blood System includes: (a) an SCBS facility or establishment licensed by the FDA to manufacture SCBS products; (b) SCBS products approved by the FDA and (c) SCBS manufacturing methods, including SOPs. The SCBS product life cycle may include six related phases or levels: (1) Patient Immunology Matching between an UCB source and/or an allogenic tissue source; (2) Receipt of source material and confirmation of patient immunology match; (3) Purification of desired stem cell subpopulation(s); (4) Ex-vivo expansion of desired stem cells; (5) Production of a Patient Treatment Kit (PTK) ready for administration; and (6) Shipment of the PTK to the transplantation center for its administration to the patient. Each of these levels of operation is governed by SOPs compliant with the Current Good Tissue Practice for Manufacturers of Human Cellular and Tissue-Based Products [hereinafter, “CGTP”] and accreditation guidelines and procedures, such as those of the American Association of Blood Banks (AABB) and the Foundation for the Accreditation of Hematopoietic Cell Therapy (FAHCT).

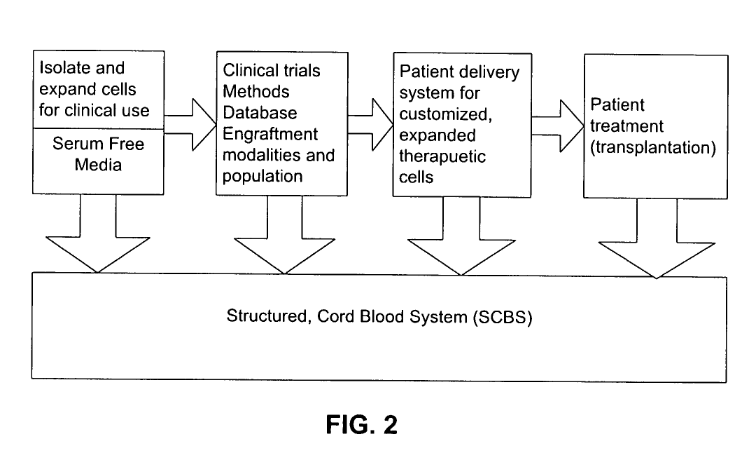

Using a system based, at least in part, on guidelines illustrated in FIG. 1, regulatory licensing compliance is managed and achieved. For example, the standards, procedures and documentations required for CGTPs, and FAHCT and AABB accreditation are built into the SCBS and product life cycle. The following further describes the levels of the SCBS product life cycle and sets forth some examples of SCBS products and procedures. FIG. 2 is a schematic diagram showing some of the processes within the structured cord blood system of the present invention.

1.0 Overall CGTP, FAHCT and AABB Compliance (SCBS Level 0)

The SCBS system complies with the applicable and current US Food and Drug Administration (FDA) statutes, regulations and proposed or draft regulations and guidelines to assure the safety and effectiveness of SCBS products. These include, but are not limited to, the Current Good Tissue Practice for Manufacturers of Human Cellular and Tissue-Based Products [hereinafter, “CGTP”]; the “Draft Document Concerning the Regulation of Placental/Umbilical Cord Blood Stem Cell products Intended for Transplantation or Further Manufacture Into Injectable Products;” “Suitability Determination for Donors of Human Cellular and Tissue-based Products;” “Establishment Registration and Listing for Manufacturers of Human Cellular and Tissue-based Products,”, and the Clinical Laboratory Improvement Amendments (CLIA), each of which are incorporated by reference herein in their entirety.

SCBS manufacturing facilities and procedures for producing umbilical cord derived stem cell products are established and followed in compliance with the appropriate CGTPs as well as the current standard procedures in the technical manuals prepared by the AABB and the FAHCT, (each of which are incorporated by reference herein in their entirety) as long as the AABB or FAHCT procedures are more stringent and consistent with the CGTP requirements. The FAHCT is a voluntary comprehensive standard-setting, inspection, and accreditation program that encompasses all phases of hematopoietic collection, processing and transplant. FAHCT has established standards for the provision of quality medical and laboratory practice in hematopoietic cell transplantation. The SCBS manufacturing facility is designed to be accredited by the AABB and the FAHCT, as well as licensed by the pertinent State health agency (e.g., Maryland Department of Health). The AABB Standards and Manuals are available through www.aabb.org; the FAHCT Standards and Manuals available through http://www.unmc.edu/Community/fahct/orders.htm. Additional pertinent standards are available from such organizations as The International Society for Hematotherapy and Graft Engineering (ISHAGE) at http://www.ishage.org/and the EMMES Corporation at http://light.emmes.com/coblt/SOP/Toc.htm.

Standard operating procedures (SOPs) are designed using standards from the FAHCT, the AABB, the National Marrow Donor Program and in accordance with current good manufacturing practices (CGMP) and the proposed rules under the CGTP. The SCBS SOPs are established and documented for the SCBS quality program according to the most stringent of the CGTP, FAHCT and AABB requirements and guidelines. They include, but are not limited to, procedures for each of the following: (1) all significant steps in the manufacture of the SCBS product, including steps used to screen and test donor UCB in order to determine their suitability; (2) the SCBS Quality Program; (3) maintenance and control of Validated SCBS Processes; (4) equipment calibration; (5) control of the in-process SCBS product; (6) verification or validation of changes to a SCBS process; (7) receipt, acceptance or rejection, distribution, and destruction or other disposition of SCBS products; (8) receiving, investigating, evaluating and documenting information from other sources; (9) SCBS facility cleaning and sanitization; (10) control and monitoring of environmental conditions at the SCBS facility; (11) cleaning, sanitizing, and maintenance of equipment at the SCBS facility; (12) receipt and verification of supplies and reagents at the SCBS facility; (13) validation and/or verification of in-house reagents; (14) the use and removal of processing material; (15) SCBS Product Labeling; (16) determining and documenting SCBS products and reagents suitable for return to inventory; (17) SCBS Records Management System; (18) SCBS Product Tracking Methods; and the (19) Review, evaluation, and documentation of all complaints. The FAHCT, and AABB standards for quality assurance include provisions for appropriate personnel qualifications and training, and record keeping.

For example, for each of the significant steps preformed in the manufacture of the SCBS products, SOPs are maintained. The procedures are designed to prevent circumstances that increase the risk of the introduction, transmission, and spread of communicable disease by ensuring that the products do not contain relevant communicable disease agents; that the products do not become contaminated during manufacturing; and that the function and integrity of the products are not impaired through improper manufacturing. At least once annually, all SCBS procedures are reviewed and, if necessary, revised, and the review is documented. SCBS facilities may adopt the CGTP compliant standard procedures in a technical manual prepared by another organization, such as the AABB or FAHCT. Obsolete procedures are archived for at least 10 years.

Establishment and Maintenance of a Quality Program

SCBS facilities establish and maintain a quality program for manufacturing the SCBS products. The SCBS quality program is designed to establish and maintain appropriate procedures to comply with CGTPs, including review, approval, revision, and archiving. Each of the SCBS manufacturing steps meets the requirements of CGTPs.

More specifically, procedures are established to: (1) receive, investigate, evaluate, and document information received from other sources; and (2) share information pertaining to the integrity and function of the SCBS product (e.g., possible contamination of the product, potential transmission of communicable disease by the product); (3) evaluate the effect adverse information about an offered or distributed product may have on the product; (4) notify the entities to whom an affected product was distributed, quarantine and recall the product; and (5) report to FDA.

SCBS procedures are also established to ensure that appropriate corrective actions and reaudits of deficiencies are taken and documented. Corrective actions are verified to ensure that they are effective and do not adversely affect the finished product. Corrective actions address the immediate problem and prevent the problem's recurrence. Documentation of corrective actions can include: (i) identifying the SCBS product affected and a description of its disposition; (ii) describing the nature of the problem requiring corrective action; (iii) describing the corrective action taken; and (iv) identifying the date(s) of the corrective action.

Procedures are also established to ensure: (1) the proper training and education of personnel; (2) establishing and maintaining appropriate monitoring systems as necessary to comply with the requirements of CGTP, such as environmental monitoring; (3) establishing and maintaining a CGTP compliant system for the maintenance of records; (4) investigating and documenting all product deviations and making CGTP required reports. Each investigation includes a review and evaluation of the product deviation, the efforts made to determine the cause, and the implementation of corrective action(s) designed to address the product deviation and prevent recurrence. The SCBS establishment performs a periodic review and analysis of all product deviations, at least once each year, for the purpose of identifying trends and adopting appropriate preventive measures. This analysis is available for review upon inspection and for submission to FDA upon request; and (5) conducting evaluations, investigations, audits, and other actions necessary to ensure compliance with the requirements of CGTPs.

The SCBS administrator has authority over and responsibility for ensuring that the quality program is effectively established and effectively maintained. The performance of the quality program is reported to the SCBS manager at least on an annual basis.

The SCBS facility conducts a comprehensive CGTP quality audit at least once annually. Special audits may also be performed. All audits are conducted in accordance with CGTP compliant procedures to assure that the quality program is operating effectively and to identify trends or recurring problems. The quality audits are conducted by individuals with sufficient knowledge, training, and experience to identify problems in the specific processes under review, but who do not have direct responsibility for the processes being audited. Any documented report of the results of the audits and reaudits, where taken is retained and reviewed by SCBS management and the management review is also documented. The SCBS establishment validates computer software for its intended use if that software is used in computers and automated data processing systems that are part of the quality program, SCBS product manufacturing or tracking system. All software changes are validated before approval and issuance and the validation activities and results are documented.

The SCBS expansion and delivery systems includes procedures related to process validation and revalidation. These include the following: transplant recipient outcome data is collected, donor recruitment is documented, all samples are tracked, licensed materials are used; for unlicensed ancillary products used, exemptions are sought; all positive infectious disease results are reported. Standard operating procedures (SOPs) describe cord blood collection source, processing, freezing, and storage, potential cord blood recipient identification, cord blood shipping, reporting of transplant recipient outcomes.

An SCBS expansion and delivery establishment maintains records of contracts, agreements, and other arrangements with other establishments under which any step in the manufacturing process is performed by the other establishment. These records include the name and address of the other establishment(s) as well as a description of each party's responsibilities.

The SCBS includes additional documentation of the following: (a)Terms and date of FDA approval for any exempted operations or alternative operations; (b) Corrective actions taken as a result of an audit of the quality program; (c) Product deviations in manufacturing SCBS products; (d) Results of all audits and reaudits of the quality program; (e) Computer validation activities and results on those computers used as part of the quality program; (f) manufacturing, or for maintaining data or records; (g) Records of the education, experience, training, and retraining of all personnel; (h) Significant facility cleaning and sanitation; (i) Environmental control and monitoring activities; (j) Equipment maintenance, cleaning, sanitizing, and calibration; (k) Receipt, verification, and use of each supply or reagent; (l) Verification and documentation of the quality of each lot of processing media used to manufacture uniquely labeled and traceable products; (m) Removal of processing material and verification activities for in-process product; (n) Changes to established processes, including rationale and the date of implementation; (o) Validation activities when the results of a process cannot be fully verified by subsequent inspection and tests; (p) Validation of any process-related claim; (q) Review and evaluation of a process and revalidation of the process, if necessary, when any changes to or deviations from a validated process occur; (r) The storage temperature of SCBS products and any corrective action taken when acceptable storage conditions are not met; (s) Receipt, acceptance or rejection, distribution, and destruction or other disposition of SCBS products; (t) The results and interpretation of all testing and screening for relevant communicable disease agents and diseases; and (u) The determination of donor suitability.

SCBS products are subject to product and establishment licensure by the FDA and require an investigational new drug (IND) exemption for use in clinical trials. SCBS products of the present invention are manufactured at a SCBS facility. Cooperative manufacturing arrangements are made between the SCBS processing laboratory and the cord blood supplier. Current good manufacturing practices (cGMP) are adhered to throughout the SCBS processes.

The SCBS integrates the following three manufacturing roles at one establishment: (i) donor-suitability determination, (ii) cell processing and (iii) product distribution. In this way, a SCBS establishment would only need to engage other establishments, under contracts, agreement, or other arrangement, to supply the source UCB and other reagents utilized in the cell processing and product distribution. The SCBS manufacturing establishment can be considered part of a cooperative manufacturing arrangement in which the SCBS manufacturer and the UCB supplier each hold product and establishment license applications. For example, human cord blood that is to be used in the manufacture of the stem cell products of the present invention will be licensed as a blood product for further manufacture and will be approved when the final expanded stem cell product is approved.

As part of the SCBS, the SCBS establishment ensures that the work performed at the engaged establishment is performed in compliance with regulatory and accreditation requirements for donor testing, screening and suitability. This is accomplished by performing periodic audits. The SCBS includes safeguards to ensure regulatory compliance throughout the manufacturing process, even where a step in the manufacturing the SCBS product, typically the UCB source supplier, is carried out at another establishment.

1.1 Patient Immunology Matching Between an UCB Source and/or an Allogenic Tissue Source (SCBS Level 1)

At a minimum, HLA typing is performed for six HLA loci, HLA-A, -B, and -DR, at low resolution/split antigen level. DNA-based testing methods are utilized for HLA-DR typing. DNA-based testing is used for HLA-A and -B. Transplant center guidelines for typing of patient, family and to confirm the HLA types of potential unrelated donors include, typing HLA-A, B, and -DR loci using primarily DNA-based testing methods at allele level resolution for DRBl and low resolution/split antigen level for HLA-A and -B. The typing of a patient and the selected donor are performed using the same set of reagents, methodology, and interpretation criteria with fresh tissue samples to ensure HLA identity. Quality assurance and quality control for HLA testing are complied to.

The SCBS of the present invention preferably includes a method and means for tracking the identity and location of each source and sample of cord blood throughout the SCBS process. The SCBS tracking method can include information on the expansion, delivery and engraftment of the SCBS product. The SCBS preferably includes a method for tracking engraftment results, including short, medium and long term engraftment results. The SCBS tracking method further includes SCBS databases having information on the individual sources of cord blood, each cord blood sample that is processed, each SCBS product that is delivered and the engraftment results for each delivered SCBS product.

In one embodiment, the SCBS includes a tracking system in which the tissue-matched cells are housed in a container means that includes an identification means. The identification means identifies the container as being associated with said cells. The tracking system can include a means for detecting the identification means as well as a means for producing an identification signal that corresponds to the identification. The tracking system may also include a means for storing and compiling the tracking signal in a tracking database.

The tracking system may also include a controller to receive the identification signal, so that the signal is compiled at the controller to form a tracking signal that is transmitted to a tracking database for each sample or product. The information contained in the signal is compiled in database together with the other information compiled with respect to each sample. The sample results may contain detailed information, including the HLA typing data, serotyping data, functional assay data, etc. Information compiled in the database can be transmitted to a monitoring site for reporting with the delivered biological product as well as to the requiring regulatory and accreditation agencies and organizations. The information contained in the SCBS tracking database may be accessed by a monitoring site at any time to determine the precise location of any given signal from any particular sample or product. The SCBS tracking system can also track engraftment results obtained from the transplant center or clinician that administers the SCBS product to the patient.

1.2 Receipt of Source Material and Confirmation of Patient Immunology Match (SCBS Level 2)

The life cycle of SCBS customized stem cell production typically begins with the request for tissue matched stem cells. CBU processing includes documentation and characterization of the donor material, including: nucleated cell count, % viability, % nucleated erythrocytes, % mononuclear cells, blood typing, flow cytometry including CD34+ phenotype assessment, hematopoietic colony formation assays, sterility assays, and immunophenotyping. When utilizing immunoabsorption beads, the total nucleated cell count is determined on an automated counter. Using the percent from the actual CD34 counter, an absolute number of CD34+ cells is determined. As genetic disease screening is available and/or pertinent, DNA based screening can be conducted. For example, RT-PCR amplification for oncogene expression, such as BCR-ABL, can be performed by known methods, such as those described by Verfaillie et al., Blood 79, 1003 (1992).

SCBS products are subject to IND regulations during clinical development and, as final biological products are subject to licensure. The cord blood stem cells are obtained as a source material for further manufacture into a final SCBS. The source material is shipped from one FDA licensed entity to the manufacturer of the SCBS product.

1.3 Purification of Desired Stem Cell Subpopulation(s) (SCBS Level 3)

Procedures are performed to purge or enrich the starting material of nucleated cell subset(s). Examples of procedures that are employed in the purification of the desired SCBS products include centrifugal elutriation, negative or positive cell selection by monoclonal antibody-based technologies, cytokine expanded cell populations, centrifugation and density gradient separation, and lysis of contaminating erythrocytes.

Various methods may be employed to enrich for the desired umbilical cord stem cells prior to their expansion. Such methods may include positive selection for CD34+ cells, such as by immunoselection using monoclonal antibodies specific for human stem cells. Examples of human stem cell selection methods include, but are not limited to those described in the following U.S. Patents and publications: 5,061,620; 5,807,686; 5,677,136; WO97/41224, 5,840,580; 5,827,742; 5,004,681 and Yin AH, et al., AC133, a novel marker for human hematopoietic stem and progenitor cells, Blood 90(12):5002-12 (Dec. 15, 1997).

The CD34+ counts are standardized by methods employed in the art. For example, a two-color or three-color assay on a single platform such as flow cytometry may be used. For example, fresh UCB nucleated cells, or expanded cells or CD34+ fraction cells can be labeled with FITC-conjugated anti-CD34, PE-conjugated anti-CD38 and cy-chrome-conjugated anti-HLA-DR, PE-conjugated CD7, and cy-chrome-conjugated CD19. Three-color antibody marker analysis can then be performed on a FACScan flow cytometry configured with the applicable sofware, such as Lysis II (Becton Dickinson). Control samples are labeled with either isotype control for FITC, FITC-conjugated anti-CD34 and/or isotype controls for PE and cy-chrome. If standard controls for CD34+ and other cells types are available to establish inter- and intra-laboratory variation for cell counts, they will be utilized.

In a preferred embodiment of the SCBS, enrichment for CD34+ cells is achieved as follows. The nucleated cells (NC) from the tissue matched UCB source are separated by sedimentation, followed by red blood cell (RBC) lysis. The UCB 34+ cells are isolated using methods known in the art, such as with a miniMACS immunomagnetic separation device using a CD34 isolation kit or CD34 multisort kit (Miltenyi Biotech Inc., Auburn, Calif.). To improve purity, cells in the CD34+ fraction can be applied to a second column and the purification is then repeated.

In another embodiment of the present invention, the enriched or expanded stem cells are altered genetically to include an exogenous DNA sequence. Such altered stem cells may be used in gene therapy.

1.4 Ex-vivo Expansion of Desired Stem Cells (SCBS Level 4)

Cell culture media that is used to grow cells that are to be introduced into a human patient, preferably does not contain ingredients such as bovine serum albumin, mammalian serum, and/or any natural proteins of human or mammalian origin. The culture medium employed in the instant invention can support CD34+ cellular proliferation and, in the presence of the appropriate ancillary proteins such as cytokine(s), expand specific cell types/lineages. The medium contains components derived from U.S. Pharmaceutical grade components that will permit it to be used in clinical regimens.

The preferable serum-free media is QBSF-60 (Quality Biological, Inc.). Preferably, the serum-free media is made fresh on the day that it is to be added to the culture. However, when storage previous to use is necessary, it may be desirable to add certain compounds. Reducing agents such as .alpha.-monothioglycerol and .beta.-mercaptoethanol, which are thought to diminish free-radical formation, may be added to the serum-free media formulations. This will enhance stability of the serum-free media during storage for lengths of time of up to 20 days or longer. Additionally, in these less than preferred circumstances, antibiotics may also be added to the media as a precaution against bacterial contamination.

All of the ingredients in the medium, including the ingredients in the basal medium, are present in amounts sufficient to support the proliferation and differentiation of CD34+ cells. The medium is formulated and sterilized in a manner conventional in the art. Typically, stock solutions of these components are made filter sterilized. A finished medium is usually tested for various undesired contaminants, such as mycoplasma or virus contamination, prior to use.

In one embodiment of the present invention, the stem cell expansion process employs serum-free medium, preferedly Quality Biological's QBSF-60 serum-free medium. QBSF-60 is described in U.S. Pat. Nos. 5,766,951 and 5,945,337, the contents of each of which are incorporated herein in their entirety.

It is preferable that the ancillary proteins used are recombinant or synthetic proteins. Most preferably, the amino acid sequence of the recombinant or synthetic protein is identical to or highly homologous with that of the human protein. Thus, the most preferable serum-free media formulations used in the SCBS process contain no animal-derived proteins and have no detectable presence of animal protein.

The cord blood-derived stem cells are expanded in serum-free media along with cytokines. The preferred cytokines include: Stem Cell Factor (SCF); FLT-3/FLK-2 ligand (FL); thrombopoietin (TPO); erythropoietin (EPO); Interleukin 3 (IL-3); Interleukin 6 (IL-6); lnterleukin 1 (IL-1); granulocyte colony stimulating factor (G-CSF); granulocyte-macrophage colony stimulating factor (GM-CSF); vascular endothelial growth factor (VEGF), and MIP1α. The more preferred cytokines include SCF, FL, TPO and IL-3. Chemokines may also be included in the media. There are over sixty chemokines, including chemotactic factors for hematopoeitic progenitor cells, such as SDF 1, CK-beta-11/CCL19, and SCL-CCL21. Other factors that may be employed include transforming growth factor (TGF) beta and tumor necrosis factor (TNF) alpha.

The cytokines may be used in various concentrations, such as those described in the art of UCB CD34+ expansion. The combination and concentration of the cytokine(s) added to the serum-free medium may vary depending on the therapeutic use of the intended stem cell product. For the production of CD34+ cells from UCB, a combination of the following cytokines may be added at defined concentrations. The concentrations of the cytokines used include, but are not limited to the following: SCF at a concentration of from about 10 to about 100 ng/ml, preferably 50 ng/ml; FL at a concentration of from about 10 to about 300 ng/ml, preferably 100 ng/ml; TPO at a concentration from about 10 to about 200 ng/ml, preferably 100 ng/ml; IL-3 at a concentration from about 5 to about 50 ng/ml, preferably at a concentration from about 10 to about 20 ng/ml; IL-6 at a concentration from about 5 to about 100 ng/ml, preferably from about 10-70 ng/ml; IL-1 at a concentration from about 5 to about 100 ng/ml, preferably 10 ng/ml; G-CSF at a concentration from about 10 to 100 ng/ml, preferably from about 25 to about 50 ng/ml; GM-CSF at a concentration from about 10 to about 100 ng/ml, preferably from about 10 to about 25 ng/ml; EPO at a concentration of from about 5 to about 100 ng/ml or from about 1 to about 10 U/ml, preferably about 6 ng/ml or 3 U/ml; MIP1 α at a concentration of from about 10 to 30 ng/ml, preferably about 20 ng/ml. The SCBS product contains expanded CD34+ cells which possess comparable clonogenic efficiencies and expansion potentials to those of the source unexpanded cord blood CD34+ cells.

The serum-free medium used, supports the proliferation and differentiation of CD34+ cells. The therapeutic regimes for which the SCBS is applied include cord blood, peripheral blood (including mobilized peripheral blood) and bone marrow transplant techniques. Most preferably, SCBS is applied to cord blood transplant techniques.

Such transplants are useful in the therapy of radiation exposure, immunodeficiency, tumors of the hematopoietic system (leukemias), genetic diseases (hemaglobinopathies, sickle cell anemia) and tissue replacement (liver schirosis, Beta islet cells). The serum-free media employed in the SCBS of the present invention can be used to cultivate mixed cell populations which contain CD34+ cells in order to selectively enrich (i.e., increase the proportion of) CD34+ cells in the population.

The serum-free medium employed in the present invention, is a formulation suitable for use in human therapeutic protocols. The media can be used in the expansion of the CD34+ cells which are responsible for repopulating the host bone marrow. The media can also be used in the expansion of these early progenitor stem cells that are transplanted as an adjunct to other therapies. Such other therapies include, but are not limited to chemotherapy, radiation therapy, immunotherapy, gene therapy, pharmaceuticals, other transplantations, including other sources of stem cells, such as fresh bone marrow or cord blood or peripheral blood. The rationale for using the expanded stern cells as an adjunct to other therapies is that the in vitro treatment allows for differentiation of the early progenitor cells to mature cells, capable of protecting the host from opportunistic diseases which occur during other therapies.

The presence of appropriate growth factors and cytokines, such as interleukins (IL), colony stimulating factors (CSF), and the like, will influence the rate of proliferation and the distribution of cell types in the population. Cytokines used for the expansion and differentiation of early progenitor cells are stem cell factor, interleukin-1 and interleukin-6. Cytokines used to stimulate proliferation and differentiation of mid-progenitor cells are interleukin-3 and granulocyte-macrophage colony stimulating factor. Cytokines which promote the differentiation of specific blood cell types are granulocyte colony stimulating factor, macrophage colony stimulating factor and erythropoietin. For hematopoietic reconstitution transplantation purposes, the GM-colony forming cells are among the most important. The myeloid population is absolutely necessary for the transplant patient to survive. The role which each of these cytokines play in hematopoiesis is under intense investigation in the art and it is expected that eventually it will be possible to faithfully recapitulate hematopoiesis in vitro.

The serum-free media employed in the present invention is suitable for storing the stem cell source cells, such as UCB, and is also particularly useful for growing the stem cells when they are removed from the human body. The serum-free medium, preferably QBSF-60, is especially adapted to selectively promote the growth of CD34+ cells so that a mixed culture of cells can be enriched in CD34 + cells so that the CD34+ cells can be administered to a patient in need of the cells. The serum-free media used in the SCBS is also useful for growing CD34+ cells after they have been separated from other cells. After the CD34+ cells have been grown to increase the number of cells, they can be given to a human patient for known therapies.

The serum-free media described in U.S. Pat. No. 5,945,337, an example of which is Quality Biologic's QBSF-60, has been optimized with U.S. Pharmaceutical grade components and is composed of the basal medium IMDM plus 2 mM L-glutamine, 100 U/ml penicillin, 100 μg/ml streptomycin, human injectable grade serum albumin (4 mg/ml) (Alpha Therapeutic Corporation), partially iron saturated human transferrin (300 μg/ml) (Sigma Chemical Corporation or Bayer Corporation) and human recombinant sodium insulin (0.48 U/ml) (Sigma). Since L-glutamine present in IMDM is unstable, additional glutamine is added later. The medium can be changed on various regimens, including but not limited to every 1-7 days, preferably every 2-7 days, more preferably every 3-7 days and most preferably every 7 days. The medium is changed often enough to allow the CD34+ cells to grow and proliferate. Unnecessary changing of the media is avoided because of extra time and expense and risk of contamination.

As was demonstrated in U.S. Pat. No. 5,945,337, the entirety of which is incorporated herein in its entirety, QBSF-60 performs superiorly in its ability to support the growth of CD34+ cells purified from umbilical cord blood, when compared to serum-containing medium (IMDM plus 20% FBS) and other serum-free media, all of which contained the cytokines SCF, IL-3, IL-6 and G-CSF at 50 ng/ml each. The other serum-free media included three commercially available serum-free media developed especially for hematopoietic cells, namely, two serum-free formulations developed especially for lymphocytes, AIM V (Life Technologies) and X-VIVO 10 (BioWhittaker) and another serum-free formulation designed especially for CD34+ cells StemPro 34 (Life Technologies). After 14 days of culture, the CD34+ cells cultured in QBSF-60 proliferate from 2×104/ml to 186-200×104/ml, whereas the CD34+ cells cultured in the serum-containing medium proliferated only from 2×104/ml to 129-134×104/ml. AIM V and X-VIVO 10 supported cord blood CD34+ cell proliferation from 1×104/ml to 49×104/ml and 123×104/ml, respectively. StemPro34 supported the proliferation of the cord blood CD34+ cells from 1×104/ml to only 88×104/ml. QBSF-60 supports the proliferation of CD34+ cells derived from umbilical cord blood to higher levels than serum-containing medium or any other commercially available serum-free medium designed especially for hematopoietic cells.

In one preferred embodiment of the stem cell expansion system of the instant invention, QBSF-60 media is utilized in the manufacturing process which is supplemented with SCF, IL-3, IL-6 and G-CSF at 50 ng/ml each in order to maintain a distinct population of cells with the immature CD34+ phenotype.

In another embodiment of the SCBS expansion system, the enriched CD34+ UCB cells are cultured in QBSF-60 (Quality Biological Inc., Gaithersburg, Md.) containing 50 ng/ml SCG, 100 ng/ml FL and 100 ng/ml TPO using incubator conditions well known in the art for human cell culture. In another embodiment of the SCBS expansion system, 20 ng/ml IL-3 may be further included in the SCG/FL/TPO cocktail. In another embodiment of the SCBS expansion system, the enriched CD34+ UCB cells are cultured in QBSF-60 containing 50 ng /ml SCG, 100 ng/ml FL, 100 ng/ml TPO.

The SCBS process may further include “ex-vivo purging” protocols, in which the source for the “normal” (non-tumorigenic) CD34+ cells is treated in vitro with reagents which are preferentially cytotoxic for the tumor cells or other undesired cell types. Alternatively, the tumor or undesired cells can be selectively depleted from the culture using immobilized antibodies which specifically bind to the undesired cell type, such as a tumor cell. The “purged” stem cell source can then be used for transplantation.

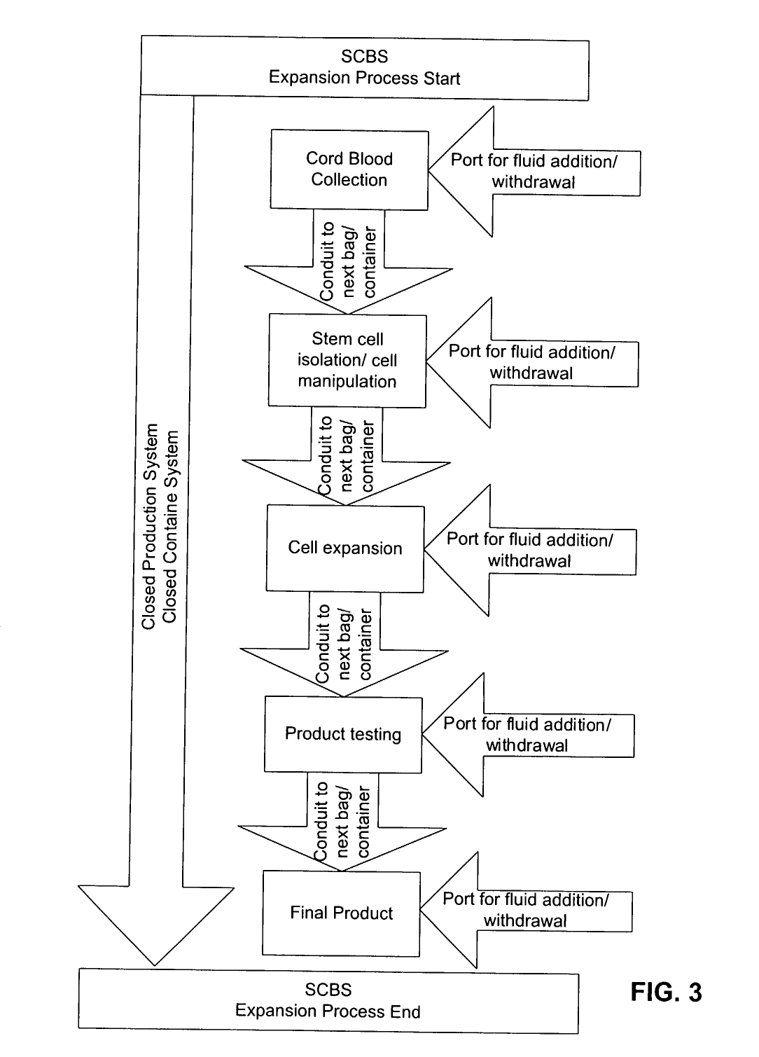

The enriched CD34+ cells can be cultured at various concentrations in a variety of culture vessels known in the art, such as flasks, bioreactors, and closed system containers. Preferably, the SCBS utilizes a closed container system. Most preferably, the closed container system employed in the SCBS of the present invention includes a container that is a Teflon bag container of the present invention, which is described below. The Teflon bag container may be 100 to 1000 ml capacity. One example of the structured cord blood expansion process of the present invention is shown schematically in FIG. 3.

In one embodiment of the present invention, the cells may be initially seeded in culture at a concentration from about 1.0×104 /ml to about 2.0×104 /ml. Preferably, 2.0×106 cells are seeded in 100 mls in a closed 100 ml Teflon container. The cells are cultured in the media for a period between day 3 and day 14, preferably between day 7 and day 14. Preferably, the cells are cultured for 7 days, and then transferred directly into a larger container having fresh media of either the same doses of cytokines or a different dose and combination of cytokines inside. Preferably, for the expansion of UCB CD34+ cells, the same cytokines of the same or similar dose are included after the seventh day in culture. The transfer preferably occurs by sterile docking and without any washing. The cells are harvested between day 7 and day 14, preferably between day 10 and day 14 and most preferably on day 14. Cell samples may be harvested at any timepoint during the expansion culture in order to test for a variety of parameters, including, but not limited to cell phenotype, sterility, viability, etc. The cell phenotypes and tests employed may include, but are not limited to, subpopulation determinations using progenitor colony-forming assays well known in the art (CFU-GM, BFU-E, CFU-GEMM, HPP-CFC, LTCIC), cell marker identification using flow cytometry or DNA analysis (CD34, CD38, CD61, CD90, HLA-DR, CD7, CD19).

A closed-system, sterilized (radiation or gas sterilization as appropriate) container system, preferably consisting of a series of appropriate containers for processing different stages of the stem-cell collection, purification, expansion production process, and with integral tubing is the preferred culture system, consisting of several separated but communicating culture containers, to be employed in the SCBS expansion system. The preferable container is a bag manufactured from Teflon or Teflon-containing components. The closed system of the present invention can provide the following advantages: (i) standardizes and facilitates the entire production process for stem cell products and to decrease outside influences on the actual process, thereby increasing CGMP compliance in a variety of environments, (ii) to facilitates the duplication of the entire SCBS process to operators in other territories, (iii) allows for easy scale-up of the SCBS process, and (iv) permits strict separation of patient-specific cell products during the entire SCBS process. One example of the closed container system of the present invention is shown schematically in FIG. 3.

In one embodiment of the present invention, the container system comprises several containers that are connected and used sequentially as the production process within the SCBS requires. The components of the closed container system include, for example, a plurality of separate but communicating containers, a label pocket, membrane ports with port covers, and integral tubing. The integral tubing can further include an injection site, male and female luer lock adapters, and roller clamps. The closed container system may further include facilities to isolate certain processing areas within the closed container system from other processing areas as to movement, temperature, light exposure, diagnostic measures (cell counts, viability assessments etc.), as well as the introduction or withdrawal of fluids and chemical agents, growth factors, and metabolic products. The label pocket allows the insertion of written information, such as the patient identification, product specifications, volume and the processing methods used. Labeling is according to the recommendations of the Standards Committee of the AABB and the FAHCT. The integral tubing set provides flexibility to use a variety of disposable transfers via syringes or a sterile connecting device. The membrane ports are available to allow for sampling, and/or the addition of other components. Aseptic fluid transfer is carried out according to accepted standards, such as in a laminar flow hood. The fill volume recommendations of the SCBS container system are based on the individual processing steps during the particular SCBS expansion process for the desired SCBS product as well as the intended use for that product. Volumes in the containers within the system range from 50 ml to 1000 ml (intervals of 50, 100, 250, 500, 750 and 1000 ml). In order to provide fresh media to the cells or harvest them, the sterile container is docked aseptically into another container. In one embodiment of the present invention, the CD34+ cells are aseptically transferred into the closed container system containing serum-free media and ancillary products.

The cellular yields, expansion efficiency, total expanded CD34+ cells and subpopulations are all documented according to SCBS Standard Operating Procedures, which are at least as stringent as CGTPs and the applicable accreditation guidelines. For example, the SCBS product is assessed and documented for the following characteristics: nucleated cells, CD45+ count, % lymphocytes of CD45+ cells (CD3/CD16 and 56/CD19/CD45 or equivalent), total number of CD34+ cells, concentration of CD34+ cells, percentage of CD34+ cells expressing CD61, CD90 or CD38, the total number of CD45+ cells and lymphocytes (CD3/CD4/CD8/CD45 or equivalent), characterization of the CD45+ subpopulations (CD3/CD16 and 56/CD45/CD19 or equivalent), total CFU-GM, total CFU-GEMM, total BFU-E. For example, the Standard Operating Procedures established for calculating the volume for colony assay. A complete description of the manufacturing process, specifications, qualification, and acceptance criteria of each ancillary product is documented.

Further assessment of SCBS product engraftment capabilities may be performed in a Human-to-Sheep Xenograft Model known in the art. See, for example, Almeida-Porada, et al., J. Hematotherapy & Stem Cell Research 9:683 (2000), which is incorporated by reference herein in its entirety. Freshly isolated or cultured CD34+ cells are injected intraperitoneally into 55- to 60-day old fetal sheep. The transplanted sheep are analyzed for donor human cell engraftment at 9 weeks after transplantation, and after birth at various timepoints. The presence of donor cells in the hematopoietic tissues of the recipient sheep (blood, marrow, liver, spleen, and thymus) is determined at intervals post-transplantation using flow cytometric analysis and hematopoietic progenitor assays. Short-term engrafting ability is examined at 60 days post-transplant, medium-term engraftment capability is analyzed at 1 week post-birth (100 days post-transplant) and long-term engraftment capability of the various cultured cell populations is analyzed at 8 months of age (335 days post-transplant).

Examples of culture methods, media and products for expansion of human stem cells include, but are not limited to those described in the following U.S. Patents and publications: U.S. Pat. No. 5,635,387; WO98/21313. Examples of expansion methods employing QBSF-60 include, but are not limited to, the following: Quality Biological brochures entitled “Products for Hematopoietic Cell Culture;” “Quality Biological Makes the Best Stem Cell Media in the World and Has the Data to Prove It;” Almeida-Porada et al., J. Hematotherapy & Stem Cell Research 9:683-693 (2000); Shadduck et al., Hematopoietic Stem Cells (Meeting Report), Stem Cells 18:154-5 (2000); Qiu, et al., J. Hematotherapy & Stem Cell Research, 8:609-618 (1999); Qiu et al., Exp Hematol 25:706 (1997); Hematology Research News 1(1): 1-2 (1997); and Brown, et al., Cancer Research Therapy and Control 7:123-129 (1998).

Other methods for expanding UCB stem cell expansion involving culture methods, media and products not utilizing QBSF-60 have been described, for example, in U.S. Pat. No. 5,635,387 and published PCT application WO98/21313. Serum free media other than QBSF-60 for UCB culture known in the art include, but are not limited to: Life Technologies Catalogue StemPro-34 serum free culture media; Capmany, et al., Short-term, serum-free, static culture of cord blood-derived CD34+ cells: effects of FLT3-L and MIP-1α on in vitro expansion of hematopoietic progenitor cells, Haematologica 84:675-682 (1999); Daley, J P, et al., Ex vivo expansion of human hematopoietic progenitor cells in serum-free StemProTM-34 Medium, Focus 18(3):62-67; Life Technologies Catalogue information on AIM V serum free culture media; BioWhittaker Catalogue information on X-VIVO 10 serum free culture media; 5,397,706 entitled Serum-free basal and culture medium for hematopoietic and leukemia cells; no cell proliferation; Kurtzberg et al., 18:153-4 (2000); Kurtzberg et al., Exp Hematol 26(4):288-98 (April 1998); http://www.aastrom.com/html/prodover.html describing trials using CB-I and CB-II Therapy Kits for cord blood; http://www.aastrom.com/html/98rel/oct30-98.htm describing the AastromReplicellTM System.

Devices, computer programs and bioreactor system for stem cell expansion are described in the following U.S. Pat. Nos. 6,096,532, “Processor apparatus for use in a system for maintaining the growing biological cells”; 6,048,721, “Bioreactor for mammalian cell growth and maintenance”; 5,994,129, “Portable cassette for use in maintaining and growing biological cells”; 5,985,653, “Incubator apparatus for use in a system for maintaining and growing biological cells; and 5,688,687, “Bioreactor for mammalian cell growth and maintenance”; Van Zant et al., Expansion in bioreactors of human progenitor populations from cord blood and mobilized peripheral blood, Blood Cells 20(2-3):482-90 (1994); http://server3.cancer.umn.edu/page/research/trsplant/cord12.html describing the University of Minnesota's clinical trial using expanded cord blood; Kobari, et al, Exp Hematol 28(12):1470-80 (2000); Yoshida et al., Br J. Haematol 98(2):254-64 (1997); Takahira et al, Ann Hematol 72(3):131-5 (1996); De Bruyn et al., Stem Cells 12(6):616-25 (1994); Yamaguchi et al., Exp Hematol 29(2):174-182 (2001); Kogler et al., Bone Marrow Transplant 21(3):233-41 (1998).

Ancillary Products Used During Production of the SCBS Products

Numerous products may be used during the production of SCBS products. The ancillary products are intended to act on the cells rather than to have an independent effect on the patient. Additionally, the intended action of the ancillary products is not dependent upon incorporation into the stem cell product with maintenance of the product's structural or functional integrity. Examples of such ancillary products to be used in the SCBS process include, but are not limited to: 1) apheresis machines; 2) equipment for purging or selecting stem cell populations; and 3) collection and storage containers.

Ancillary products also include reagents that are not intended to be present in final products. Some of the ancillary products used in the SCBS expansion system are already regulated under an existing IND, NDA, PLA, PMA, or premarket notification, such as QBSF-60. Other ancillary products used in the present invention are regulated under drug or device CGMP's, such as recombinant human (rh) EPO, rh-SCF, rh-IL-1 beta, rh-transforming growth factor (TGF) beta, rh-tumor necrosis factor (TNF) alpha, rh-TPO. The ancillary products that are used during the manufacturing process are described under the IND for the final hematopoietic stem cell product. Complete descriptions of the use of the ancillary product in the manufacturing process are provided.

Non-ancillary products are those that are administered directly to a patients or a product whose function requires incorporation into the cord blood stem cell product with maintenance to some degree of structural or functional integrity. Such products are regulated as drugs or biological products. Examples include, but are not limited to: 1) anticoagulants added to the collection container and infused with the product into the recipient; and 2) storage medium and cryoprotective agents added to the stored product and infused with the product into the recipient.

Other methods of expanding cord blood stem cells have been described, which are incorporated by reference herein in their entirety: Civin, Stem Cells 18:150 (2000); Novelli et al, Hum Gene Ther 10(18):2927-40 (1999); Sakabe et al., Eur J. Haematol 60(5):297-306 (1998); Fisher, et al., Current Problems in Obstetrics Gynecology and Fertility 19(2):75 (1996); Piacibello et al., Blood 89:2644-2453 (1997); Koller et al., Bone Marrow Transplant 21 (7):653-63 (1998); Bhatia et al., J. Exp Med 186(4):619-24 (1997);Laver J et al., Exp Hematol 23(14):1492-6 (1995); Almici et al., Haematologica 80(5):473-9 (1995); Gilmore, et al., Exp Hematol 28(11): 1295-305 (2000); Pecora A L et al., Bone Marrow Transplant 25(7):797-9 (2000); Nakahata, Rinsho Byori, Suppl 110:54-62 (1999); Querol et al., Bone Marrow Transplant 21 Suppl 3:S77-80 (1998); U.S. Pat. No. 5,610,056 entitled Use of stem cell factor interleukin-6 and soluble interleukin-6 receptor to induce the development of hematopoietic stem cells; U.S. Pat. No. 5,599,703 entitled In vitro amplification/expansion of CD34+ stem and progenitor cells; U.S. Pat. No. 5,670,351 entitled Methods and compositions for ex vivo replication of human hematopoietic stem cells w/stable genetic transformation; WO97/17079 entitled Method of allogeneic hematopoietic stein cell transplantation without graft failure or graft vs. host disease; U.S. Pat. No. 5,646,043 entitled Methods for ex vivo replication of human stem cells and/or expansion of human progenitor cells; WO00/36090 entitled Human brain endothelial cells and growth medium and method for expansion of primitive CD34+ CD38− bone marrow stem cells; U.S. Pat. No. 5,541,103 entitled CD34+ peripheral blood progenitor cells obtained by ex vivo expansion; U.S. Pat. No. 5,399,493 entitled Methods and compositions for the optimization of human hematopoietic progenitor cultures; and U.S. Pat. No. 6,030,836 entitled In Vitro maintenance of hematopoietic stem cells.

1.5 Patient Treatment Kit (PTK) Ready for Administration (SCBS Level 5)

SCBS products that are made available for release are capable of maintaining their function and integrity, are not contaminated, and do not contain communicable disease agents. The SCBS products are defined in terms of the cell dose/number (in terms of total nucleated cells, CD34+ cells) HLA typing for HLA-A, -B, and -DR. FDA licensure is likely to be based on cell dose and HLA mismatch. The clinician decides the units and phenotype that is acceptable for transplantation. Persons responsible for progenitor cell infusion are the clinicians who order the cells.

SCBS procedures maintain the function and integrity of the SCBS product. Additional SCBS procedures make the SCBS product available for distribution by providing release criteria designed to prevent the release of any products that may be in quarantine, contaminated, deteriorated, or from donors who have been determined to be unsuitable or for whom a donor-suitability determination has not been completed. Before making a SCBS product available for distribution, the SCBS procedures verify and document that the release criteria have been met and review all records pertaining to the SCBS product. The determination that the SCBS product is available for distribution is documented and dated.

SCBS is capable of producing a somatic cell product that is licensed by the FDA and approved for particular indications. Such a product is: (1) enriched and expanded from a source of a minimum collected volume of 30 mls of umbilical cord blood which was collected from an accredited (e.g., FACT/NETCORD approved) laboratory; (2) processed and cryopreserved according to Accredited standards; (3) sterile; and (4) labeled for RH and ABO typing, HLA typing and the A, B, and DR-beta-1 loci, and post-processing counts, CD34+ counts, CFU-GM counts, infectious disease screening, family history and evidence of maternal consent for donation. The SCBS product ordered for transplant would include cells that match a minimum of 4/6 antigens or 3/6 alleles, a cell dose of a minimum of 2×107 cells/kg post-processing count for patients who are less than 12 years or less than 50 kg or a minimum cell dose of 1×107 cells/kg for patients over 12 years of age or over 50 kg.

Tests for Infectious Diseases Transmissible by Blood

The SCBS product is tested for fungal, bacterial and viral infectious diseases transmissible by blood. The typical infectious agents transmitted in stem cell transplants have been documented in the literature. For example, see Webb et al., Transfusion, vol. 36:782 (1996); Price et al., American Journal of Respiratory Critical Care Medicine, 158:876 (1998); Espinosa et al., Transfusion, 36:789 (1996); Kogler et al., Journal of Hematotherapy, 5:105 (1996). Examples of such diseases include, but are not limited to the following: CMV, EBV, HIV, Hepatitis A, B, and C, and micoplasma. Examples of the tests for such infectious disease agents that can be performed include, but are not limited to, those cited in the above cited publications, which are incorporated by reference herein, as well as microbiological culture, antibody or antigen markers, DNA chip analysis, Real Time PCR (RT-PCR).

SCBS Product Compositions

The SCBS production methods produce SCBS compositions for clinical use are comprised of particular documented quantities and qualities of expanded CD34+ cells. Such quantities and qualities include: at least 100 million CD34+ stem cells from a single umbilical cord source; at least 20 million CFU-GM from a single umbilical cord source; a quantity of CD34+ stem cells from a single umbilical cord source sufficient for single and/or multiple engraftments in adults; hematopoietic stem cells possessing clonogenic efficiencies and expansion potentials that are comparable to those of the unexpanded cord blood CD34+ cells. The SCBS products of the inventions are used for transplantation in humans, preferably recipients larger than 50 kgs and adults.

1.6 Shipment of the PTK to the Transplantation Center for Its Administration to the Patient (SCBS Level 6)

A fundamental aspect of the SCBS is that the SCBS establishment distributes the SCBS product directly to the transplantation center that ordered a patient specific product. The SCBS method most preferably includes the step of distributing the SCBS product. The distributing step may use the distribution infrastructure of commercial courier services or an service within the SCBS. By distributing the regulated SCBS product, the SCBS method optionally further includes steps for conducting and/or documenting the operating procedures necessary to ensure that the SCBS product is manufactured in compliance with all applicable regulations and accreditation standards.

SCBS Packaging and shipping containers are designed, validated, and constructed to ensure that the SCBS product function and integrity are protected from damage, deterioration, contamination, or other adverse effects during customary conditions of processing, storage, handling, and distribution.

SCBS Product Label

The SCBS products of the present invention may be used in a variety of treatment modalities. Such methods include transplantation to replace the hematopoietic compartment, including particular dysfunctional cell types of the hematopoietic system. Examples of human stem cell treatments include, but are not limited to those described in the following U.S. Patents and publications: U.S. Pat. No. 5,914,108 entitled Human hematopoietic stem cell; WO99/30723 entitled Use of Human Umbilical Cord Blood for Adoptive Therapy; Brichard et al., Persistence of fetal hemoglobin production after successful transplantation of cord blood stem cells in a patient with sickle cell anemia J Pediatr 128(2):241 (1996); Miniero et al., Bone Marrow Transplant 22 Suppl1:S78-9 (1998); Kelly et al., J Pediatr 130(5):695 (1997); http://www.stem-cell.com describing umbilical cord blood cell transplant services; and http://www.itxm.org/CBB/hsc.htm describing ltxM Diagnostics Hematopoietic Stem Cell Laboratory.

All publications, patents and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Conclusion

Embodiments of systems and methods for managing the life cycle of SCBS product manufacturing have been described. In the foregoing description, for purposes of explanation, numerous specific details are set forth to provide a thorough understanding of the present invention. It will be appreciated, however, by one skilled in the art that the present invention may be practiced without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the present invention. Additional advantages and novel features of the invention forth in the description, and in part will become apparent to those skilled the art or upon examination of the detailed description or may be learned by practice of the invention. The detailed description shows the preferred embodiment of the invention by way of illustration of the best mode contemplated for carrying out the invention. In the detailed description, systems and methods in accordance with embodiments of the present invention have been described with reference to specific exemplary embodiments. The present invention is capable of other and different embodiments, and its several details are capable of modifications in various obvious respects, all without departing from the scope and spirit of the present invention. Accordingly, the drawings and descriptions are to be regarded as illustrative in nature, and not as restrictive.

What is claimed is:

1. A stem cell manufacturing system for delivering tissue-matched stem cells, the system comprising: a delivery system for delivering a biological product comprised of tissue-matched stem cells and a stem cell expansion system that produces a biological product, said stem cell expansion system being coupled to the delivery system.

2. The stem cell manufacturing system of claim 1 wherein said delivery system receives the patient order.

3. The stem cell manufacturing system of claim 1 wherein said delivery system identifies a suitable source of donor blood.

4. The stem cell manufacturing system of claim 3, wherein said donor blood is umbilical cord blood.

5. The stem cell manufacturing system of claim 1, wherein the tissue-matched stem cells are CD34+ cells.

6. The stem cell manufacturing system of claim 1, wherein the tissue-matched stem cells are matched by HLA loci, said HLA loci selected from the group consisting of, HLA-A, HLA-B, and HLA-DR.

7. The stem cell manufacturing system of claim 1, wherein the tissue-matched stem cells are CFU-GM cells.

8. The stem cell manufacturing system of claim 1, wherein said biological product is licensed.

9. The stem cell manufacturing system of claim 1, wherein said delivery system is conducted at a licensed establishment.

10. The stem cell manufacturing system of claim 1, wherein said stem cell expansion system is conducted at a licensed establishment.

11. The stem cell manufacturing system of claim 1, wherein said delivery system and stem cell expansion system are conducted at the same licensed establishment.

12. The stem cell manufacturing system of claim 1, wherein said biological product is ready for administration to the patient.

13. The stem cell manufacturing system of claim 1, wherein said tissue-matched stem cells are CD34+ cells that have been expanded more than 200-fold.

14. The stem cell manufacturing system of claim 1, wherein the tissue-matched stem cells are matched using methods selected from the group consisting of: DNA-based testing methods and low resolution/split antigen level.

15. The manufacturing system of claim 14, wherein said matching is HLA loci matching using methods selected from the group consisting of: DNA-based testing methods and low resolution/split antigen level.

16. The manufacturing system of claim 15, wherein the tissue-matched stem cells possess at least three HLA loci identical to the HLA loci of the patient order.
